(19) United States
(12) Patent Application Publication
(10) Pub. No.: US 2008/0073572 A1
(43) Pub. Date: Mar. 27, 2008

(54) Systems and methods of measuring power in lithography systems

(76) Inventors: Siegfried Schwarzl, Neubiberg (DE); Stefan Wurm, Austin, TX (US)

Correspondence Address: SLATER & MATSIL LLP, 17950 PRESTON ROAD, SUITE 1000, DALLAS, TX, 75252, US

(21) Appl. No.: 11/490,007

(22) Filed: Jul. 20, 2006

(51) Int. Cl. A61N 5/00

(52) U.S. Cl. 250/492.2

(57) ABSTRACT

Systems and methods of measuring power in lithography systems are disclosed. A preferred embodiment comprises a metrology method that includes providing a lithography system and measuring an amount of power of the lithography system using the Compton effect.

### TECHNICAL FIELD

The present invention relates generally to lithography systems used to manufacture semiconductor devices, and more particularly to the measurement of power in lithography systems.

### BACKGROUND

Generally, semiconductor devices are used in a variety of electronic applications, such as computers, cellular phones, personal computing devices, and many other applications. Home, industrial, and automotive devices that in the past comprised only mechanical components now have electronic parts that require semiconductor devices, for example.

Semiconductor devices are manufactured by depositing many different types of material layers over a semiconductor workpiece, wafer, or substrate, and patterning the various material layers using lithography. The material layers typically comprise thin films of conductive, semiconductive, and insulating materials that are patterned and etched to form integrated circuits (ICs). There may be a plurality of transistors, memory devices, switches, conductive lines, diodes, capacitors, logic circuits, and other electronic components formed on a single die or chip, for example.

For many years in the semiconductor industry, optical lithography techniques such as contact printing, proximity printing, and projection printing have been used to pattern material layers of integrated circuits. Optical photolithography involves projecting or transmitting light through a pattern comprised of optically opaque or translucent areas and optically clear or transparent areas on a mask or reticle. Lens projection systems and transmission lithography masks are used for patterning, wherein light is passed through the lithography mask to impinge upon a photosensitive material layer disposed on semiconductor wafer or workpiece. After development, the photosensitive material layer is then used as a mask to pattern an underlying material layer.

There is a trend in the semiconductor industry towards scaling down the size of integrated circuits, to meet the demands of increased performance and smaller device size. For lithographic printing of integrated circuit patterns having a size of about 50 mn or less, extreme ultraviolet (EUV) lithography is under development, which uses light in the soft x-ray range, e.g., having a wavelength of about 10 to 15 nm. In EUV lithography systems, reflective lenses and masks are used to pattern a photosensitive material layer disposed on a substrate, for example.

Because of the short wavelength used in EUV lithography systems, EUV power cannot be easily measured by diverting a small fraction of the beam to monitor and control the exposure dose, as it is currently measured in lithography tools and systems that utilize visible light in the deep ultraviolet (DUV) range, e.g., having wavelengths of about 248 nm to 193 nm, for example. EUV lithography systems typically utilize a wavelength of about 13.5 nm, which is easily absorbed by prior art exposure dose test methods, for example.

Thus, what are needed in the art are improved methods and systems for measuring power in EUV lithography systems.

### SUMMARY OF THE INVENTION

These and other problems are generally solved or circumvented, and technical advantages are generally achieved, by preferred embodiments of the present invention, which provide novel systems and methods of measuring power in lithography systems, wherein the Compton effect is used to measure photon energy or electron energy to determine the power of the lithography system.

In accordance with a preferred embodiment of the present invention, a metrology method includes providing a lithography system, and measuring an amount of power of the lithography system using the Compton effect.

The foregoing has outlined rather broadly the features and technical advantages of embodiments of the present invention in order that the detailed description of the invention that follows may be better understood. Additional features and advantages of embodiments of the invention will be described hereinafter, which form the subject of the claims of the invention. It should be appreciated by those skilled in the art that the conception and specific embodiments disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is a schematic illustrating a Compton scattering process in momentum space in accordance with an embodiment of the present invention;

FIG. 2 is a graph showing the change of photon wavelength after collision with an electron at rest;

FIG. 3 is a graph showing the change of photon wavelength after collision with an electron at various kinetic energies;

FIG. 4 shows a wavelength spectrum of an EUV beam emitted from an EUV source;

FIG. 5 is a graph of the absolute value of the maximum wavelength shift of EUV photons scattered from electrons as a function of the electron kinetic energy;

FIG. 6A is a polar plot in x-y coordinates for the scattering of EUV photons with electrons of 50 k eV kinetic energy;

FIG. 6B illustrates the angle δ between the x-y and ξ-η coordinate systems shown in FIG. 6A;

FIG. 7 shows the angle δ as a function of the initial kinetic energy of the incident electron;

FIG. 8 shows the correlation of the electron scattering angle θ and the photon scattering angle φ for various electron kinetic energies;

FIG. 9 is a polar plot of the kinetic energy of scattered electrons in the ξ-η coordinate frame for electrons having an initial energy of 10 eV;

FIG. 10 illustrates normalized kinetic electron energy in dependency of electron scatter angle θ and initial kinetic electron energy;

FIG. 11 shows normalized kinetic electron energy in dependency of the electron scatter angle θ for different initial kinetic electron energies;

FIG. 12 shows an EUV source/collector module of an EUV lithography system in accordance with an embodiment of the present invention;

FIG. 13 is a block diagram of a system and method for measurement of EUV intensity in accordance with a first embodiment of the present invention;

FIG. 14 is a block diagram of a system and method for measurement of EUV intensity in accordance with a second embodiment of the present invention;

FIG. 15 is a block diagram of a system and method for measurement of EUV intensity in accordance with a third embodiment of the present invention;

FIG. 16 is a schematic that may be implemented in the third embodiment shown in FIG. 15;

FIG. 17 is a block diagram of a system and method for measurement of EUV intensity in accordance with a fourth embodiment of the present invention; and

FIG. 18 is a block diagram of a system and method for measurement of EUV intensity in accordance with a fifth embodiment of the present invention.

Corresponding numerals and symbols in the different figures generally refer to corresponding parts unless otherwise indicated. The figures are drawn to clearly illustrate the relevant aspects of the preferred embodiments and are not necessarily drawn to scale.

### DETAILED DESCRIPTION OF ILLUSTRATIVE EMBODIMENTS

The making and using of the presently preferred embodiments are discussed in detail below. It should be appreciated, however, that embodiments of the present invention provide many applicable inventive concepts that can be embodied in a wide variety of specific contexts. The specific embodiments discussed are merely illustrative of specific ways to make and use the invention, and do not limit the scope of the invention.

The need for EUV power measurement techniques will first be described, followed by a description of some concepts used in embodiments of the present invention, a description of some preferred embodiments of the present invention, and some advantages thereof.

In an EUV lithography system, variations in EUV power need to be measured at several different critical locations along the optical beam path. The relative measurements then need to be combined to determine an EUV power stability measurement. In addition to monitoring the stability of EUV power levels at several locations in an EUV exposure system, it is also important to correctly identify sources of EUV power variations, such as fluctuations in generated per pulse power, variation of EUV power due to transmission losses through the system in the source, the illuminator, at the mask level, at individual projection optics, or at the wafer level.

One of the critical locations where EUV power needs to be monitored is at what is referred to in the art as the intermediate focus (IF), which is defined as the clean EUV photon point at the exit aperture of the source/collector module. The clean photon specification at IF means that only EUV photons with a given wavelength variation around a central wavelength (typically a 1.75 to 2% bandwidth specification around a wavelength of 13.5 nm) are present, for example.

However, the measurement of EUV photon power at IF is difficult and is currently only accomplished in an off-line mode, not during actual productive use of a lithography system. In current methods of measuring EUV photon power at IF, detectors are used that block the beam pass to collect EUV photons. Such detectors cannot be used for an in-situ measurement to measure EUV power and EUV power fluctuations during actual use of the lithographic system for wafer exposure.

In addition, EUV lithography systems include devices to deflect EUV light into detectors that utilize mirrors or gratings, which are susceptible to contamination; therefore such devices need frequent re-calibration.

What are needed in the art are methods for monitoring EUV power that do not interfere with the productive use of an EUV lithography system, and in particular, that do not degrade the imaging quality or wafer throughput, e.g., by reducing the amount of EUV light that arrives at the wafer level. In-situ methods of measuring EUV power that may be implemented when an EUV lithography system is being used to fabricate or process semiconductor wafers are needed in the art.

The present invention will be described with respect to preferred embodiments in a specific context, namely for measurement of power in EUV lithography systems. Embodiments of the invention may also be applied, however, to measurement of power in other lithography applications and other types of lithography systems, for example.

Embodiments of the present invention involve measuring and monitoring EUV power during productive use of an EUV lithography system without impacting lithographic tool performance using the Compton effect. An electron beam is used to intersect the EUV beam at the IF or other locations in the optical path of the EUV lithography system, and the number of EUV photons scattered out of the EUV beam into a particular angular range or the number of electrons scattered out of the electron beam into a particular angular range are measured. By choosing particular angular ranges of the scattered photons or electrons and particular energies of the incident electrons, the performance of the EUV intensity measurement can be optimized (e.g., with respect to the signal-to-noise ratio). Embodiments of the present invention comprise using the Compton effect to measure intensities of short wavelength light to control exposure dose in lithography tools.

The Compton effect, also referred to as Compton scattering, describes the scattering process of an electron with a photon. The Compton effect restricted to scattering of photons by electrons at rest is described by Semat, H., et al., in “Introduction to Atomic and Nuclear Physics,” Fifth Ed., 1972, pp. 142-153, Holt, Rinehart and Winston, Inc., New York, N.Y., which is incorporated herein by reference.

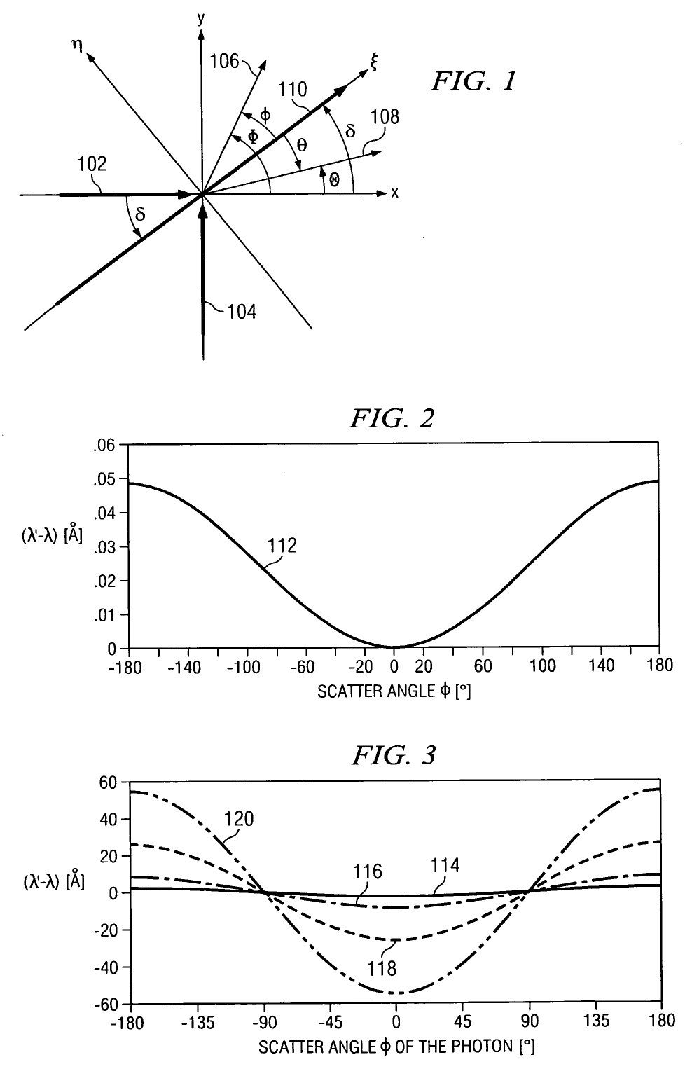

FIG. 1 is a schematic illustration of a Compton scattering process in momentum space considering electrons of arbitrary momentum γmv. The Compton scattering process in the momentum space is shown, wherein the x-y Cartesian coordinate system defines the “laboratory frame” or coordinates used for test purposes. A photon, represented by vector 102, enters the momentum space along the negative x-axis with an energy of hν and a momentum of hν/c, where h is Planck's constant, c is the velocity of light, and ν is the frequency of the photon 102. An electron, represented by vector 104, enters the momentum space along the negative y-axis with an energy of mc²(γ−1) and a momentum of γmv, where m is the mass of the electron 104, v is the velocity of the electron 104 and γ=(1−v²/c²)^{1/2}. After the collision of the photon 102 and electron 104 at the origin of both the x-y coordinate system and the origin of the ξ-η coordinate system, the photon is scattered, as shown at 106, and the electron is scattered, as shown at 108.

Adding the two momentum vectors 102 and 104 defines the total momentum 110 which is equal to ((hν/c)²+(γmv)²)^{1/2}. The total momentum 110 is conserved in the scattering process; e.g., the total momentum vector 110 has the same length and direction before and after the scattering process. The total momentum vector 110 also defines the ξ-axis of the ξ-η coordinate system, as shown. The x-y Cartesian coordinates refer to the laboratory frame, and the η-ξ coordinates refer to the rotated frame in which the ξ-axis is aligned with the total momentum vector 110.

Using energy and momentum conservation in the ξ-η coordinate system yields Equations 1, 2, and 3:

$$\begin{matrix}hv + {mc}^{2} ( γ - 1 ) = {hv}^{′} + {mc}^{2} ( {γ}^{′} - 1 ) ; & Eq . 1 \text{:} \\ P = \frac{hv}{c} z = \frac{{hv}^{′}}{c} cos φ + {γ}^{′} {mv}^{′} cos θ ; & Eq . 2 \text{:}\end{matrix}$$

wherein

$$z = {[ 1 + {( \frac{γ mvc}{hv} )}^{2} ]}^{1 / 2} ,$$

and P is the total momentum vector 110; and

$$0 = \frac{{hv}^{′}}{c} sin φ - {γ}^{′} {mv}^{′} sin θ . \qquad Eq . 3 \text{:}$$

In FIG. 1, the angles φ and θ refer to the scattering angle of the scattered photon 106 and the scattered electron 108, respectively, with respect to the total momentum vector 110. The “primed” variables in the equations refer to their values after the scattering process. The angles Θ and Φ define the scattered electron 108 and scattered photon 106 with respect to the x axis. Angle δ is the angle between the momentum vector 110 and the x axis, and the angle δ is also the angle between the x-y coordinate system and the ξ-η coordinate system.

From Equations 1 through 3, the wavelength change of the photon λ′-λ and the photon momentum after the collision Pₚₕ, can be calculated, as shown in Equations 4 and 5.

$$\begin{matrix}{λ}^{′} - λ = \frac{{λ}_{c}}{γ} ( 1 - z cos φ ) ; and & Eq . 4 \text{:} \\ {P}_{{ph}^{′}} = \frac{{hv}^{′}}{c} = \frac{hv / c}{1 + \frac{hv}{γ {mc}^{2}} ( 1 - z cos φ )} ; & Eq . 5 \text{:}\end{matrix}$$

wherein λ_{c}=h/mc is the Compton wavelength of the electron.

The kinetic energy of the scattered electron 108 is shown in Equation 6:

$${E}_{kin} = {mc}^{2} ( γ - 1 ) + \frac{hv α}{γ} \cdot \frac{1 - z cos φ}{1 + \frac{α}{γ} ( 1 - z cos φ )} ; \\ wherein α = \frac{hv}{{mc}^{2}} . \qquad Eq . 6 \text{:}$$

The scatter angle φ of the photon 106 and the scatter angle θ of the electron 108 with respect to the ξ-η coordinate system are correlated to each other according to Equation 7:

$$cot θ = \frac{1}{sin φ} [ z ( 1 + \frac{α}{γ} ) - ( 1 + {z}^{2} \frac{α}{γ} ) cos φ ] . \qquad Eq . 7 \text{:}$$

Equations 4 through 7 are mathematical solutions of the conservation Equations 1 through 3 for both positive angles φ (0°≦φ≦18°) and negative angles φ(−180°≦φ>0°). However, the scattering cross-sections in dependency of the scatter angles φ and θ remain unknown. Based on physical experience it can be assumed that scattering into negative angular ranges of φ and θ should be unlikely compared to those for positive ones. The optimum angles and angle ranges for the particular measuring set-up may be determined experimentally, for example.

FIG. 2 is a graph 112 showing the change of photon wavelength after collision with an electron at rest, according to Equation 4. The change of photon wavelength (λ′-λ) in Angstroms Å vs. the scatter angle φ in degrees is shown after collision with an electron at rest, wherein before the collision, the electron has a kinetic energy of 0 eV. In FIG. 2, the photon wavelength λ before scattering is 13.5 nm. In FIG. 2, it can be seen that a photon scattering from an electron at rest loses energy, and the wavelength of the scattered photon is increased by a small amount, e.g., less than or equal to about 0.005 nm).

FIG. 3 shows the change of photon wavelength according to Equation 4 for the scattering of a photon of 13.5 nm wavelength with electrons that have kinetic energy values ranging from 100 eV to 50 keV, respectively. The change of photon wavelength (λ′-λ) after collision with electrons at various kinetic energies before the collision is shown. The photon wavelength λ before scattering is 13.5 nm. Results for an electron having a kinetic energy of 100 eV is shown at 114, an electron having a kinetic energy of 1 keV is shown at 116, an electron having a kinetic energy of 10 keV is shown at 118, and an electron having a kinetic energy of 50 keV is shown at 120.

In FIG. 3, a photon scattered from an electron with 100 eV kinetic energy shows a significantly larger shift in wavelength, with the maximum change around 0.27 nm for back scattering (e.g., for −180°>φ>−90° and 90°≦φ≦180°, the photon loses energy) as well as for forward scattering (e.g., for −90°≦φ≦90°, the photon gains energy). In contrast to photon scattering by electrons at rest, both positive and negative shifts in wavelength result, that increase with increasing energy of the incident electrons.

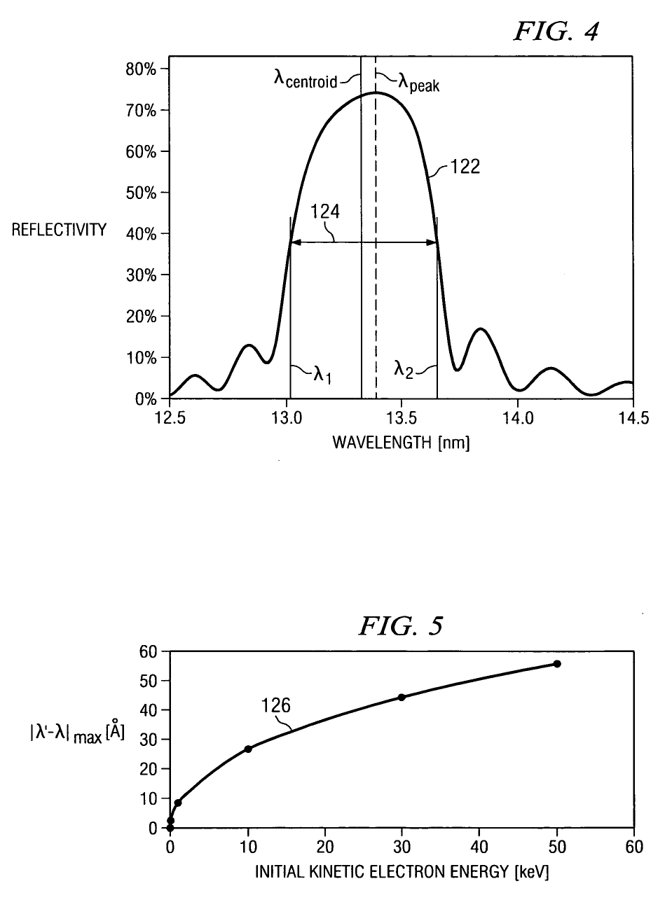

For EUV lithography tools and systems, the requirements for EUV light are typically a 1.75% to 2.00% bandwidth around the central wavelength of 13.5 nm, for example. A typical EUV light spectrum 122 of a lithography source is shown in FIG. 4. As can be seen from the narrow EUV photon wavelength spectrum in FIG. 4 (e.g., at a Full Width Half Maximum (FWHM) 124 of about 0.7 nm), a shift in the order of one nanometer in wavelength for forward or back scattered photons is clearly distinguishable from photons that are not scattered by using suitable wavelength discriminating devices. The reflectivity at the centroid wavelength λ_{centroid}=(λ₁+λ₂)/2 is due to the spectrum asymmetry below the maximum reflectivity at λₚₑₐₖ, as shown in FIG. 4.

FIG. 5 is a graph 126 of the absolute value of maximum wavelength shift |λ′-λ|ₘₐₓ in Å of EUV photons scattered from electrons as a function of the electron kinetic energy. The absolute maximum wavelength shift of scattered EUV photons increases with the kinetic energy of the electrons. For electrons having a kinetic energy of 100 eV, the maximum wavelength shift is about 0.27 nm, which shifts the maximum of the spectral distributions of scattered photons from the centroid wavelength λ_{centroid} of about 13.4 nm shown in FIG. 4 to about 13.1 nm (forward scattering) or to about 13.6 nm (back scattering). If even higher electron kinetic energy values are used, e.g., greater than or equal to about 1 keV, the wavelength of the scattered photons with maximum wavelength change completely shifts out of the wavelength band shown in FIG. 4. Such larger wavelength shifts make it easier to discriminate the scattered photons spectrally from the photons arriving at the IF, and the signal-to-noise ratio rises correspondingly, for example.

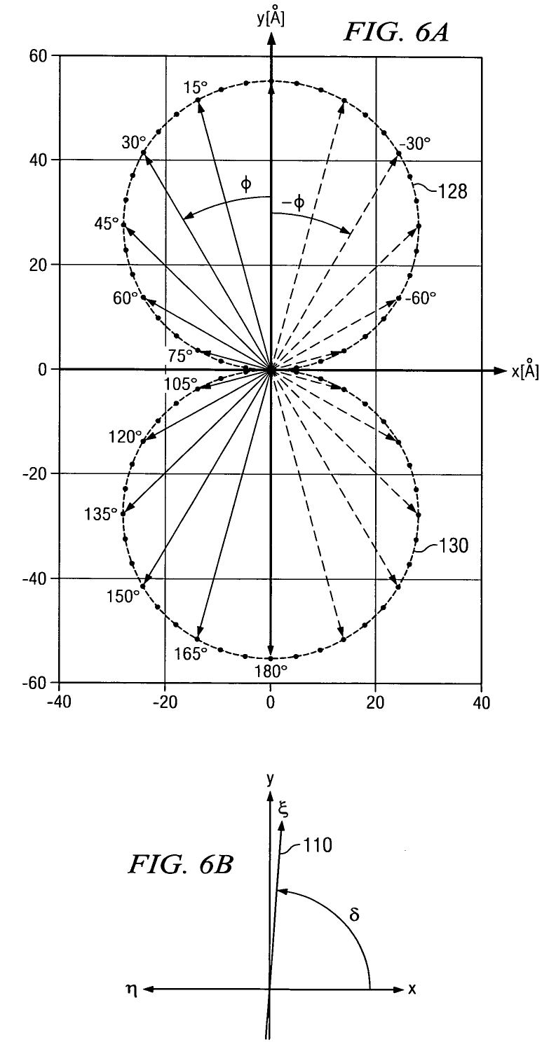

In the case of sufficient signal-to-noise ratio, the scattered photons can be measured in the entire angular range from φ=0° to φ=180°. FIG. 6A is a polar plot in x-y coordinates (the laboratory coordinates) for the scattering of EUV photons with an electron of about 50 keV kinetic energy. FIG. 6A shows the angular distribution of the wavelength change (λ′-λ) for EUV photons (λ=13.5 nm) in the scattering plane in a polar plot for incident electrons of 50 keV. The case where λ′ is less than λ is shown at 128, and the case where λ′ is greater than λ is shown at 130. The lengths of the arrows represent the magnitudes of the wavelength changes. For these particular parameters, the angle between the x-axis and the total momentum vector of the photon and electron is δ=89.98°, as shown in FIG. 6B at 110. Therefore, the x-y system and the ξ-η coordinate system coincide within the drawing accuracy. In the case shown in FIG. 6A, the maximum wavelength changes are about 5.6 nm for scattering angles φ close to 0° or 180°, for example.

In accordance with embodiments of the present invention, detectors may be used to measure scattered photons or electrons, utilizing the Compton effect to quantify the EUV power of an EUV lithography system. For example, a detector may be set up to capture scattered photons 106 into a solid angle ranging from close to zero to about 2π, for example. The total number of detected photons 106 is proportional the total number of EUV photons 102 arriving at the IF or other point along the optical path, for example. Highly sensitive large area detectors may be used to collect as many scattered EUV photons 106 as possible, for example. To measure EUV photons 102, an electron beam 104 may be used to separate a small fraction of photons 106 from the incoming photon beam 102, for example. The fraction of scattered photons 106 is preferably small, e.g., preferably less than about 1% of the incoming photons 102, in accordance with embodiments of the present invention, so that a lithography system may be used for exposure of semiconductor wafers during the metrology tests to measure the EUV photons 102 described herein, for example. The number of scattered photons 106 detected per time unit is proportional to the number of incident EUV photons, provided that the characteristics of the electron beam 104 (such as, as examples, energy, current, and cross-section) are kept constant.

Thus, in summary, when scattered photons 106 are used to measure the incident EUV radiation intensity in accordance with some embodiments of the present invention, then the use of high current and high energy electron sources are preferable, e.g., that operate at about 1 to 100 microamperes and at least about 1 kilovolt, because they create not only high photon scattering intensities, but also maximum wavelength shifts that increase with the electron energy, thus allowing the application of wavelength-sensitive discrimination techniques. The optimum electron beam current depends on the cross-section of the electron beam that may range from nm to mm dimensions for measuring EUV intensities either very locally or an average over a larger volume.

In contrast, when scattered electrons 108 are used for measuring the EUV radiation intensity in accordance with other embodiments of the present invention, then the use of low energy electron sources are preferable, e.g., that operate also at about 1 to 100 microamperes but at lower voltages of about 50 to 300 volts.

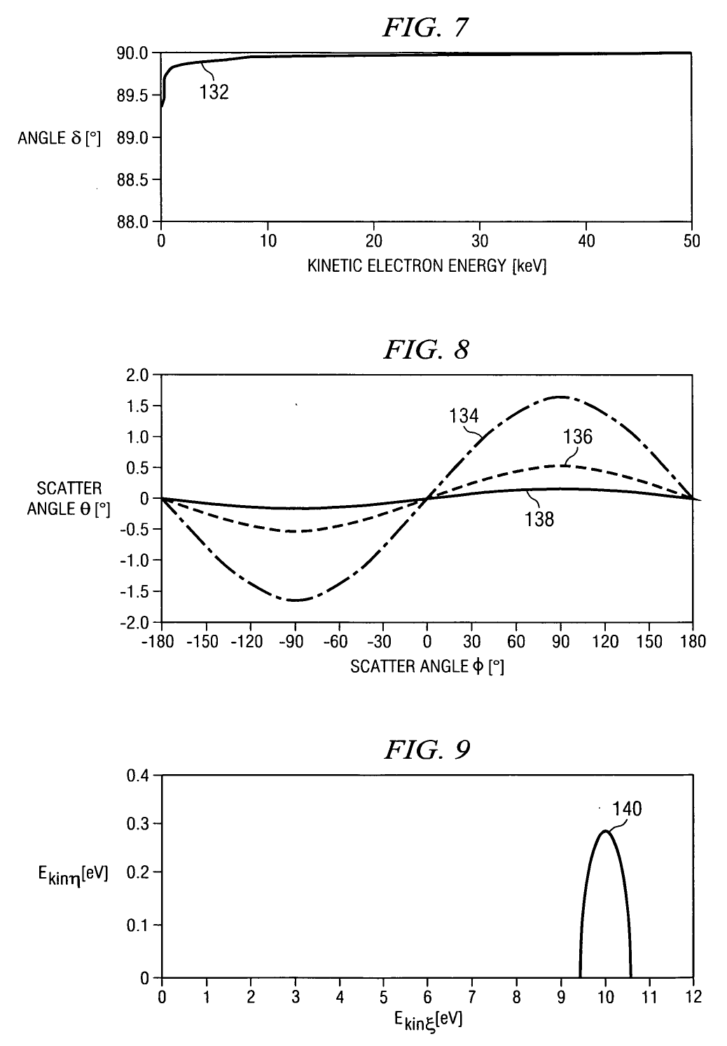

FIG. 7 shows the angle δ as a function of the initial kinetic energy of the incident electron at 132, and FIG. 8 shows the electron scattering angle θ and the photon scattering angle φ, which are correlated by Equation 7. In FIG. 8, the relationship between electron scattering angle θ and photon scattering angle φ is shown for three electron kinetic energy values of 10 eV at 134, 100 eV at 136, and 1,000 eV at 138. FIG. 8 illustrates that the electron scattering angle θ as a function of the photon scattering angle φ is about: |θ|<1.65° for 10 eV; |θ|<0.52° for 100 eV; and |θ|<0.17° for 1,000 eV, as examples.

With increasing electron energy, the angle δ approaches 90°, as shown in FIG. 7, and the scatter angle θ of the electrons decreases or narrows, as shown in FIG. 8. For example, the spatial separation of the scattered electrons 108 from the incident electrons 104 becomes increasingly difficult to detect as the kinetic energy is increased.

For higher electron kinetic energy values, the electron scattering angle θ decreases rapidly. However, for lower electron kinetic energy values, the electron scattering angle θ is large enough and the angle δ is sufficiently separated away from 90° that the scattered electrons signal can be separated from the unscattered electron signal or electron beam.

FIG. 9 shows a polar plot of the kinetic energy components of scattered electrons in the ξ/η coordinate frame at 140 for an initial electron energy before scattering of 10 eV. As can be seen, for the initial electrons, the change in kinetic energy in ξ- direction can be up to about +0.59 eV for photons ±180° back scattered, or up to about −0.56 eV for photons scattered forward at 0°.

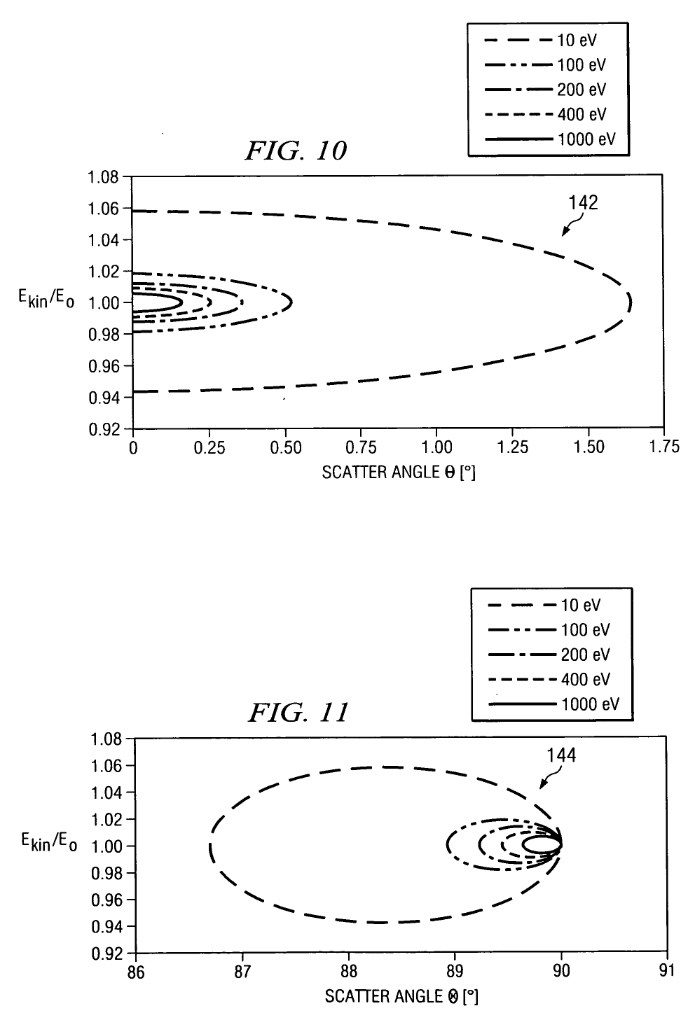

FIG. 10 illustrates the normalized kinetic electron energy in dependency of electron scatter angle θ in degrees and initial kinetic electron energy E₀ in eV at 142. FIG. 11 illustrates the normalized kinetic electron energy in dependency of the electron scatter angle θ in degrees for different initial kinetic electron energies in eV at 144. FIG. 11 shows that the separation of the scattered and incident electrons is easiest for low electron energy values, because first, the scatter angles θ are larger for low electron energies; and second, at high electron energies the direction of the scattered electrons θ approach that of the unscattered electrons (e.g., wherein Θ=90°).

Embodiments of the present invention use Compton scattering for deriving a measurable signal so that the EUV radiation intensity in EUV lithography using 13.5 nm wavelength can be measured at IF or other locations in the path of illumination energy (e.g., the optical path) of an EUV lithography system during the productive use of the lithography tool or system. In some embodiments, the number of scattered photons 106 (see FIG. 1) per unit time is collected across a certain solid angle as a monitor of EUV power at IF. This number of scattered photons 106 per time unit into a certain angular range is proportional to the number of EUV photons of fixed energy (13.5 nm) incident per time unit and thus to the EUV radiation power. The wavelength shift of scattered photons 106 is used to optimize the detection of scattered photons 106, improving the signal-to-noise ratio of the scattered photon 106 intensity measurements.

In other embodiments, electrons 108 scattered out of the incoming electron beam, e.g., beam 104 in FIG. 1, are used as a signal to monitor EUV power at IF. The measured change in electron kinetic energy of the scattered electrons 108 is used to improve the detection of the scattered electrons 108, improving the signal-to-noise ratio of scattered electron current measurement.

Embodiments of the present invention provide methods of measuring EUV power in an operating lithography tool without introducing bulky instruments and mirrors into the beam path, which would prevent the productive use of the tool. The measurement means comprises an electron beam having a diameter of between a few nanometers and a few millimeters and is therefore applicable to measure EUV intensities averaged over these dimensions. The diameter of the electron beam 104 may comprise about 1 nm to about 5 mm, as examples, although other diameters may be used. The electron beams are highly transparent for EUV light, and do not distinctly disturb the optical path. The measuring methods described herein can also be used at other wavelengths than used in EUV lithography, e.g., and can be used in other types of lithography systems.

Five preferred embodiments of the present invention will next be described. In the embodiments described herein, exemplary methods and systems for measuring the intensity or power of the incident EUV radiation are shown.

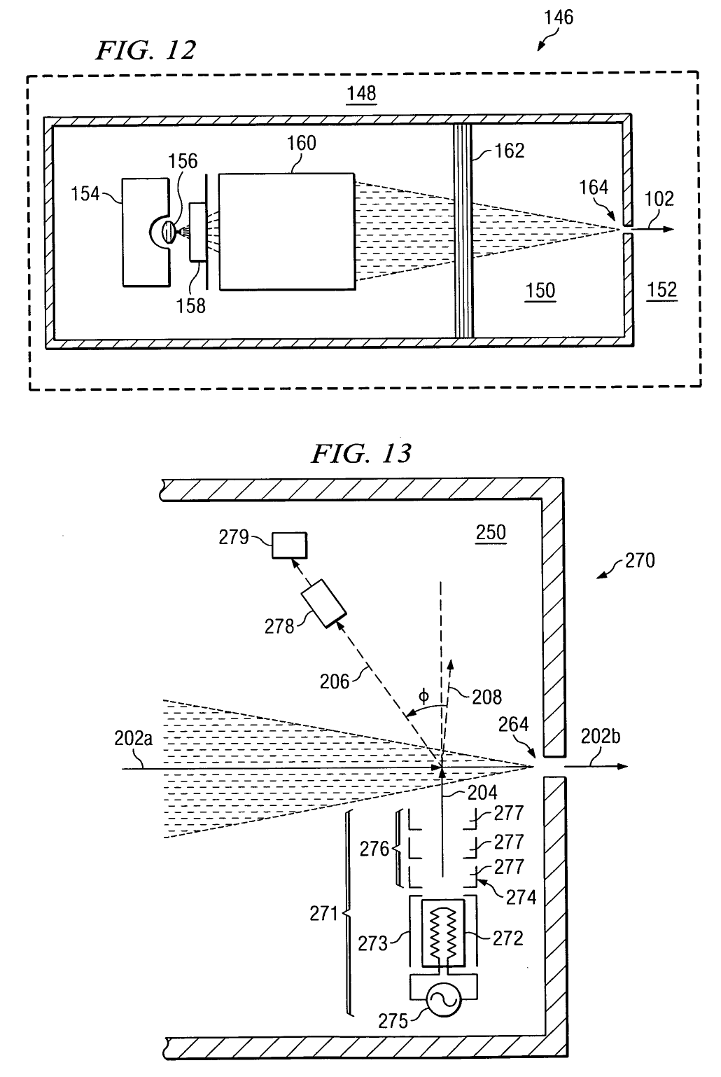

Referring next to FIG. 12, an EUV source/collector module 146 of an EUV lithography system in accordance with an embodiment of the present invention is shown. The diagram illustrates the various necessary elements of a source/collector module 146 used to generate ‘clean photons’ 102 at the intermediate focus IF, e.g., at an aperture 164. The wavelength of an EUV lithography system is typically 13.5 nm and the size of the IF aperture 164 is in the order of about 1 to 10 millimeters, depending on the maximum source size the illuminator can adjust, e.g., the etendue of the system.

The source/collector module 146 includes a source 154 adapted to produce plasma 156 proximate a debris mitigation device 158 which is proximate a collector 160. The collector 160 outputs a field of photons that passes through a spectral purity filter 162. The photons 102 pass through the IF aperture 164 that separates the source side 150 from the illuminator side 152 of the source/collector 146. The photons 102 exit the aperture 164 on the illuminator side 152, as shown. The entire source/collector module 146 is typically contained in a vacuum 148, as shown.

Embodiments of the present invention are preferably implemented in the source side 150 of a source/collector module 146, although alternatively, the novel methods of measuring intensity of energy used for lithography systems may be implemented anywhere in the optical path of a lithography system, for example, e.g., on the illuminator side 152 or elsewhere in the optical path (not shown).

FIG. 13 is a block diagram of a system 270 and method for measurement of EUV intensity in accordance with a first embodiment of the present invention. The source side 250 of a source/collector module is shown in FIG. 13. The incident photon beam 202a is shown on the left side of the IF aperture 264. The IF aperture 264 comprises an exit aperture of the source/collector module, for example.

In accordance with embodiments of the present invention, an electron source 271 is provided that generates an incident electron beam 204. The electron source 271 may include a cathode 272 comprising an alkali oxide at a zero potential contained within a Wehnelt cylinder 273 at a negative potential (e.g., about −1 keV, an anode 274 at positive potential (e.g., about +1 keV), and a current source 275 coupled to the cathode 272. The electron source 271 is adapted to define the kinetic energy of the primary electrons emitted by the electron source 271, for example. The electron source 271 includes an electrostatic lens 276 comprising at least one electrode 277, e.g., about three electrodes, with an outer electrode at a positive potential (e.g., about +1 keV) and an inner electrode at negative potential (e.g., about −1 keV). The electron source 271 may comprise other devices adapted to produce an electron beam 204, for example. It may be advantageous in this embodiment (and also in the following embodiments) for the electron source 271 to be spatially separated from the source side of the source/collector module, and for only a small orifice in the separating wall to be used to allow the electron beam 204 to enter the source/collector module, for example. Under these conditions, it will be easier to fulfill different vacuum conditions (e.g., pressures) of the source/collector module and the electron source, for example.

On an opposite side of the incident photon beam 202a from the electron source 271, a detector 278 is included, which preferably comprises a photon detector, in this embodiment. An amplifier 279 may be coupled to the detector 278 to amplify the signal detected, e.g., the deflected photons 206. Additional electronics for storing and processing the information gathered may be included in the test system 270 or may be coupled externally to the test system 270, for example (not shown).

Advantageously, the majority of the photons 202b exit the IF aperture 264 undisturbed after the measurement. Therefore, the test system 270 may be used during the productive use of a lithography system or tool. The scattering angle φ previously described herein between the scattered photons 206 and the ξ-axis (e.g., the direction of the total momentum vector 110 in FIG. 1 or P in Eq. 2) is shown in FIG. 13, for example. For realistic electron energies (e.g., ≧about 1 keV), the direction of the ξ-axis differs only by a small angle (≦about 0.05°) from that of the scattered electrons). Advantageously, the entire angular range of scattered photons 206 from φ=0 to φ=180° may be used to measure the intensity of the incident photon beam 202a. The angle φ may comprise a range of angles that are optimized to measure the scattered photons 206, for example.

The photon 206 detection can be widely adjusted by choosing the detector 278 position and the size of the detector 278 window. The amplified detector 278 signal can be used as measurement for the power, e.g., the dose, of the lithography system, e.g., the EUV power of an EUV lithography system.

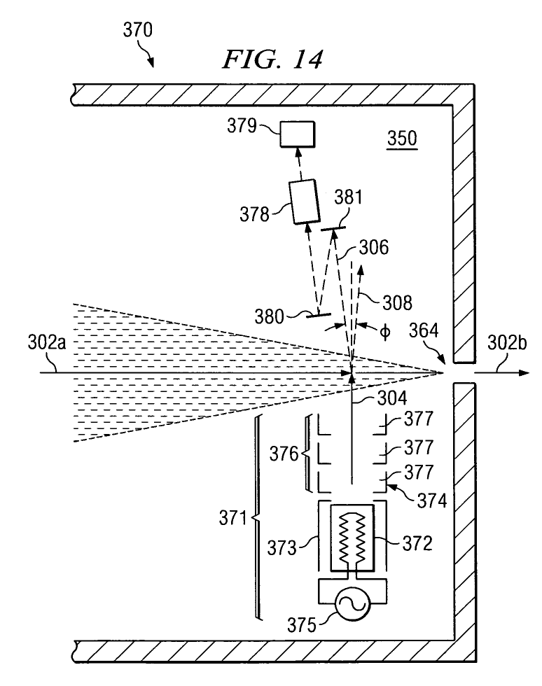

FIG. 14 is a block diagram of a system 370 and method for measurement of EUV intensity in accordance with a second embodiment of the present invention. Like numerals are used for the various elements that were used to describe the previous figures. To avoid repetition, each reference number shown in FIG. 14 is not described again in detail herein. Rather, similar materials x02a, x02b, x04, x06 . . . are preferably used for the various elements and components shown as were used in the previous figures, where x=1 in FIGS. 1 through 12, x=2 in FIG. 13, and x=3 in FIG. 14. As an example, the preferred and alternative materials and dimensions described for the electron source 271 in the description for FIG. 13 may also be used for the electron source 371 of FIG. 14.

In this embodiment, a plurality of mirrors 380 and 381 are used to deflect and redirect the scattered photons 306, as shown. The mirrors 380 and 381 preferably comprise multi-layered mirrors that are tuned to the reflected wavelength of the scattered photons 306, for example. The plurality of mirrors 380 and 381 may be adapted to adjust the angle φ of detection of the deflected photons 306, for example. Again, as in the first embodiment, the photons 306 comprise a signal that is detected and used to quantify the amount of power of the lithography system.

Mirror 381 may comprise a multilayer mirror comprising a plurality of alternating types of materials, such as Mo and Si, for example, tuned to the wavelength of the reflected photons 306, for example. Mirror 380 may comprise a multilayer mirror tuned to the same wavelength as mirror 381, for example.

Again, in this embodiment, the entire angular range of scattered photons 306 from φ=0 to φ=180° may be used to measure the intensity of the incident photon beam 302a. For angles φ close to 0° and 180°, the largest wavelength changes, e.g., up to Δλ of about 5.5 nm for 50 keV electron energy) exist (e.g., refer again to FIG. 3). The narrow bandwidths of both the incident EUV beam 302a and the two-fold reflected scattered EUV beam 306 by the mirrors 380 and 381 in this embodiment allow a highly wavelength selective measurement with high signal-to-noise ratio by strong suppression of the initial EUV beam 302a background level. The mirrors 380 and 381 are adapted to adjust a wavelength of detection of the deflected signal, e.g., the deflected photons 306, for example.

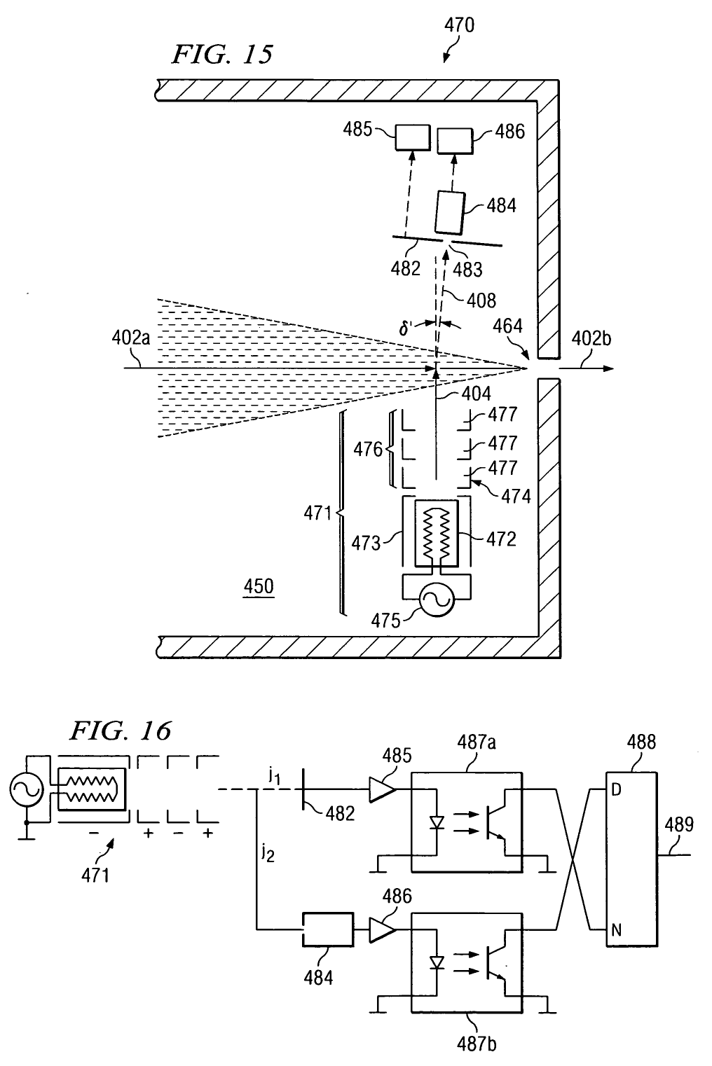

FIG. 15 is a block diagram of a system and method for measurement of EUV intensity in accordance with a third embodiment of the present invention. FIG. 16 is a schematic that may be implemented in the third embodiment shown in FIG. 15. Again, like numerals are used for the various elements that were used to describe the previous figures, and to avoid repetition, each reference number shown in FIGS. 15 and 16 is not described again in detail herein.

In this embodiment, rather than measuring deflected photons, deflected electrons 408 are measured at angle δ′. An electron source 471 comprising similar components described for the embodiments shown in FIGS. 13 and 14 may be used, although preferably, higher voltages are used than in the previous embodiments described herein. For example, the Wehnelt cylinder 473 is preferably at a negative potential of about −80 V, and the anode 474 is preferably at a positive potential of about +200 V, which defines the kinetic energy of the primary electrons 404. The outer electrode of the electrostatic lens 476 is preferably at positive voltage of about +200 V, and the inner electrode is preferably at negative potential of about −200 V, as examples.

In this embodiment, a detector adapted to measure the deflected electrons 408 is used. For example, the detector may include an electrode 482 adapted to measure the unscattered electron current. An aperture 483 may be disposed in the electrode 482, which may comprise a diameter of about 200 μm, as an example. The detector may include a Faraday cup 484 on an opposite side of the aperture 483 in the electrode 482 from the deflected electron beam 408, wherein the detector is adapted to measure the electrons 408 deflected by an angle δ′. The detector may include one or more amplifiers 485 and 486, as shown. Amplifier 485 may be coupled to the electrode 482 and may comprise a long integration time amplifier, for example. Amplifier 486 may be coupled to the Faraday cup 484 and may comprise a more sensitive amplifier. Because, in general, pulsed EUV sources are typically used in lithography systems, phase-sensitive amplifiers may be used: for example, amplifier 486 may comprise a lock-in amplifier.

A schematic of the test system 470 shown in FIG. 15 is shown in FIG. 16. The schematic includes two opto-couplers 487a and 487b coupled to the amplifiers 485 and 486, respectively. The opto-couplers 487a and 487b are coupled to an electronic divider 488, as shown. The current j₁ comprising the non-deflected electron or e-beam current is measured by the electrode 482, and the current j₂ comprising the deflected pulsed e-beam current is measured by the Faraday cup 484. The currents j₁ and j₂ are compared to determine the amount of the electron beam 408 deflected, thus providing an indication of the power of the photon beam 402a.

For example, in this embodiment, the opto-couplers 487a and 487b are integrated between the electrode 482 and the amplifier 485, and between the Faraday cup 484 and amplifier 486, respectively, in order to galvanically separate the electrode 482 and the Faraday cup 484 from the electronic divider 488 and other electronics 489. This allows the electrode 482 and the Faraday cup 484 to be placed at the same high potential as the last lens electrode potential (e.g., at about 10 k eV). Thus, the electron beams 404 and 408 are not subject to deflection due to zero potential difference between the last lens electrode and the electrode 482 and the Faraday cup 484. Electronic division of the scattered electron current j₂ by the unscattered electron current j₁ results in a signal S=j₂/j₁ that is independent of source current fluctuations.

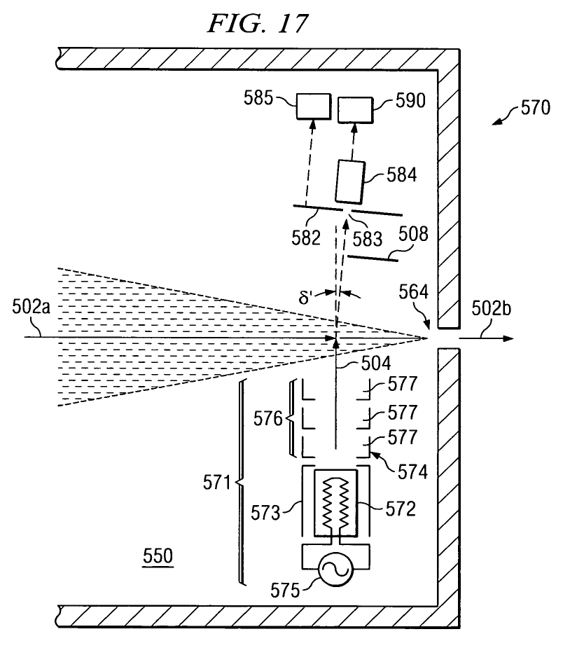

FIG. 17 is a block diagram of a system and method for measurement of EUV intensity in accordance with a fourth embodiment of the present invention. This embodiment is similar to the embodiment shown in FIG. 15, except that for small currents of scattered electrons 508, amplifier 486 of FIG. 16 is replaced by an electron counter and counting rate to voltage converter 590. The electron counter/counting rate to voltage converter 590 may comprise a low-pass integration circuit, for example.

Low energy electron beams are preferably used for the third and fourth embodiments shown in FIGS. 15 and 17, and thus, they may easily be deflected by external electric and magnetic fields. Measures have therefore to be taken to shield the electron beam from these fields. For example, Faraday cages for electric fields and high magnetic susceptibility shields for magnetic fields may be included, not shown.

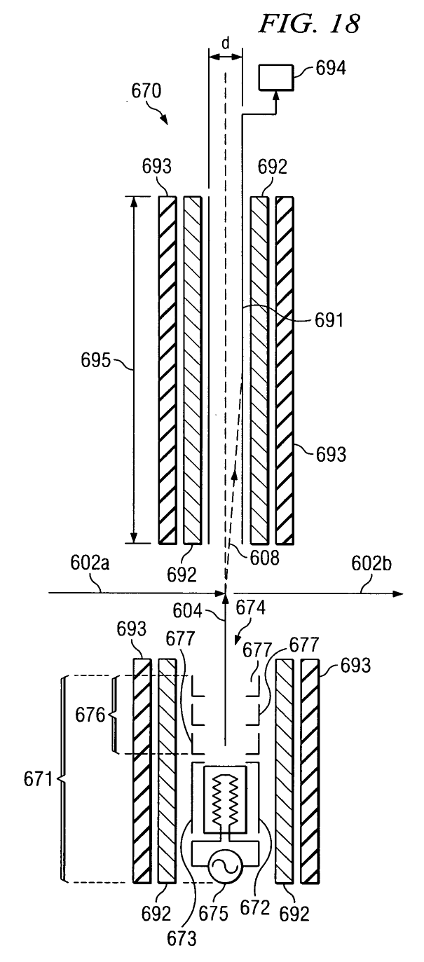

FIG. 18 is a block diagram of a system and method for measurement of EUV intensity in accordance with a fifth embodiment of the present invention. This embodiment includes a tube electrode 691 for the measurement of the scattered electron current 608. The axis of the tube electrode 691 is preferably substantially aligned with the center direction of the unscattered electrons. The diameter d and the length 695 of the tube electrode 691 are preferably chosen such that the ratio of d/length 695 is both large enough so that the unscattered electrons pass the tube 691, and small enough so that most of the scattered electrons 608 are captured by the wall of the tube, for example. This trade-off depends on the divergence of the unscattered electron beam and the power dependent scattering angles of the electrons, for example.

The scattered electrons 608 are collected by the tube electrode 691. Further amplification and conversion of the tube current may be similar to that described for the third and fourth embodiments shown in FIGS. 15 and 17, respectively, for example. An amplifier 694 may be coupled to the scattered electron beam signal 608 collected, as shown. In order to protect low-energy electrons, e.g., having a kinetic energy of less than about 1 keV from deflections caused by external magnetic and electric field fluctuations, shielding by outer tubes of high magnetic permeability and of conducting material may be included, such as magnetic shielding 692 and electric shielding 693, as shown. The electron source 671 may also include magnetic shielding 692 and electric shielding 693, for example, to prevent performance degradation when operated at low voltages used to provide a low energy electron beam 604.

In FIG. 18, the electron source 671 is preferably used having relatively low voltages. For example, the Wehnelt cylinder 673 is preferably at a negative potential of about −80 V, and the anode 674 is preferably at a positive potential of about +200 V, which defines the kinetic energy of the primary electrons 604. The outer electrode of the electrostatic lens 676 is preferably at positive voltage of about +200 V, and the inner electrode is preferably at negative potential of about −200 V, as examples. Alternatively, other voltage levels may be used, for example.

Embodiments of the present invention include metrology methods and test systems, and lithography systems implementing and including the metrology methods and systems described herein.

Embodiments of the present invention also include semiconductor devices manufactured using the novel lithography systems and test methods of power described herein, and methods of manufacturing semiconductor devices. For example, in accordance with a preferred embodiment, a method of fabricating a semiconductor device includes providing a semiconductor device having a layer of photosensitive material disposed thereon, providing a lithography system, measuring an amount of power of the lithography system using the Compton effect, and affecting the layer of photosensitive material of the semiconductor device using the lithography system. The lithography system preferably includes a photon source, an electron source proximate the photon source, and a detector for measuring a signal deflected by the electron source, wherein measuring the amount of power of the lithography system comprises measuring the signal deflected to determine an amount of power of the photon source of the lithography system.

Electrons are directed from the electron source towards photons emitted from the photon source, and the deflected signal may be measured to determine the amount of power of the lithography system, while patterning the layer of photosensitive material of the semiconductor device with the lithography system, in some embodiments. In other embodiments, the deflected signal may be measured to determine the amount of power of the lithography system before, during or after patterning the layer of photosensitive material of the semiconductor device with the lithography system, for example.

The semiconductor device may comprise a first semiconductor device, and the power of the lithography system may be adjusted during or after measuring the deflected signal. Then, a layer of photosensitive material of a second semiconductor device may be patterned using the lithography system with adjusted power. The power adjustments described herein may be made instantaneously, by the use of feedback loops, for example.

The layer of photosensitive material may be disposed over a material layer to be patterned of the semiconductor device. Affecting the layer of photosensitive material of the semiconductor device may comprise patterning the layer of photosensitive material to expose portions of the material layer to be patterned, and affecting the material layer of the semiconductor device is preferably through the patterned layer of photosensitive material. Affecting the material layer may comprise implanting the material layer with a substance, etching the material layer, forming a material or the material layer, or other manufacturing process steps, as examples.

Advantages of embodiments of the present invention include providing source metrology and dose control for lithography systems such as EUV lithography systems. The Compton effect is used to measure intensities of short wavelength light to measure and control the exposure dose in lithography tools. Advantageously, an electron source is used to direct electrons towards photons emitted from a lithography source. Deflected electrons or photons are then measured to determine the power or dose of the photons emitted from the source, using embodiments of the invention described herein. Advantageously, only a small amount of the photons are deflected so that the measurements may be made while a lithography system is being used, e.g., to expose a layer of photosensitive material on a semiconductor device. Thus, a real-time method of measuring and monitoring power and dose of exposure energy is achieved.

Although embodiments of the present invention and their advantages have been described in detail, it should be understood that various changes, substitutions and alterations can be made herein without departing from the spirit and scope of the invention as defined by the appended claims. For example, it will be readily understood by those skilled in the art that many of the features, functions, processes, and materials described herein may be varied while remaining within the scope of the present invention. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A metrology method, comprising:
providing a lithography system; and
measuring an amount of power of the lithography system using the Compton effect.

2. The metrology method according to claim 1, wherein the lithography system is adapted to output a first beam of energy, wherein measuring the amount of power of the lithography system comprises outputting the first beam of energy from the lithography system, directing a second beam of energy towards the first beam of energy, and measuring an effect of directing the second beam of energy on the first beam of energy or the second beam of energy.

3. The metrology method according to claim 2, wherein outputting the first beam of energy from the lithography system comprises outputting a photon beam, and wherein directing the second beam of energy comprises directing an electron beam.

4. The metrology method according to claim 2, wherein measuring the amount of power of the lithography system comprises measuring an amount of electrons or an amount of photons deflected after directing the second beam of energy towards the first beam of energy.

5. The metrology method according to claim 1, wherein providing the lithography system comprises providing an EUV lithography system including a photon source and an electron source.

6. The metrology method according to claim 1, wherein measuring the amount of power of the lithography system is implemented at an intermediate focus (IF) of the lithography system or other location along an optical path of the lithography system.

7. A metrology method, comprising:
providing a lithography system, the lithography system comprising a source adapted to emit a beam of photons;
directing a beam of electrons towards the beam of photons;
measuring a deflected beam from directing the beam of electrons towards the beam of photons; and
analyzing the deflected beam to determine an intensity of the beam of photons emitted from the source of the lithography system.

8. The metrology method according to claim 7, wherein directing the beam of electrons towards the beam of photons causes deflection of a portion of the beam of electrons, and wherein measuring the deflected beam comprises measuring the deflected portion of the beam of electrons.

9. The metrology method according to claim 8, further comprising using a change in electron kinetic energy of the deflected portion of the beam of electrons to improve detection of the deflected portion of the beam of electrons, improving a signal-to-noise ratio of the measurement of the deflected portion of the beam of electrons.

10. The metrology method according to claim 7, wherein directing the beam of electrons towards the beam of photons causes deflection of a portion of the beam of photons, and wherein measuring the deflected beam comprises measuring the deflected portion of the beam of photons.

11. The metrology method according to claim 10, further comprising using a wavelength shift of the deflected portion of the beam of photons to optimize detection of the deflected portion of the beam of photons, improving a signal-to-noise ratio of the measurement of the deflected portion of the beam of photons.

12. The metrology method according to claim 7, further comprising determining an optimum angle of deflection at which to measure the deflected beam, and measuring the deflected beam at the optimum angle of deflection determined.

13. A method of fabricating a semiconductor device, the method comprising:
providing a semiconductor device having a layer of photosensitive material disposed thereon;
providing a lithography system;
measuring an amount of power of the lithography system using the Compton effect; and
affecting the layer of photosensitive material of the semiconductor device using the lithography system.

14. The method according to claim 13, wherein the lithography system includes a photon source, an electron source proximate the photon source, and a detector for measuring a signal deflected by the electron source, wherein measuring the amount of power of the lithography system comprises measuring the signal deflected to determine the amount of power of the photon source of the lithography system.

15. The method according to claim 14, wherein the signal deflected comprises deflected photons and wherein the electron source is adapted to operate at about 1 to 100 microamperes and at least about 1 kilovolt; or wherein the signal comprises deflected electrons and wherein the electron source is adapted to operate at about 1 to 100 microamperes and about 50 to 300 volts.

16. The method according to claim 14, wherein the electron source comprises a current source, a Wehnelt cylinder disposed within a cathode, an anode, and an electrostatic lens.

17. The method according to claim 14, further comprising directing electrons from the electron source towards photons emitted from the photon source, and measuring the deflected signal to determine the amount of power of the lithography system, either while, before, or after patterning the layer of photosensitive material of the semiconductor device.

18. The method according to claim 17, wherein the semiconductor device comprises a first semiconductor device, further comprising adjusting the power of the lithography system after measuring the deflected signal, and patterning a layer of photosensitive material of a second semiconductor device using the lithography system after adjusting the power.

19. The method according to claim 13, wherein the layer of photosensitive material is disposed over a material layer to be patterned of the semiconductor device, wherein affecting the layer of photosensitive material of the semiconductor device comprises patterning the layer of photosensitive material to expose portions of the material layer to be patterned, further comprising affecting the material layer of the semiconductor device through the patterned layer of photosensitive material.

20. A semiconductor device patterned using the method according to claim 19.

21. A lithography system, comprising:
an illuminator including a source of photons, wherein the illuminator is adapted to direct photons along an optical path of the lithography system;
an electron source proximate the optical path of the lithography system; and
a detector for measuring a deflected signal generated by electrons directed from the electron source towards photons directed from the illuminator.

22. The lithography system according to claim 21, wherein the detector comprises a photon detector or an electron detector.

23. The lithography system according to claim 21, further comprising at least one amplifier coupled to an output of the detector.

24. The lithography system according to claim 21, wherein the detector includes a plurality of mirrors adapted to adjust a wavelength of detection of the deflected signal.

25. The lithography system according to claim 21, wherein the detector comprises an electrode for measuring unscattered electron current, and a Faraday cup opposite an aperture in the electrode from the electron source for measuring a deflected signal comprising scattered electrons.

26. The lithography system according to claim 25, further comprising a first amplifier coupled to the electrode and a second amplifier coupled to the Faraday cup, a first opto-coupler coupled to an output of the first amplifier, a second opto-coupler coupled to an output of the second amplifier, and an electronic divider for comparing a first output of the first amplifier with a second output of the second amplifier.

27. The lithography system according to claim 25, further comprising an electron counter and counting rate voltage converter proximate the deflected signal.

28. The lithography system according to claim 21, wherein the detector comprises a tube electrode.

29. The lithography system according to claim 28, further comprising magnetic shielding and/or electric shielding disposed on the tube electrode and/or the electron source.
